(19) United States
(12) Patent Application Publication
(10) Pub. No.: US 2026/0125464 A1
(43) Pub. Date: May 7, 2026

(54) METHODS OF TREATING CACHEXIA

(71) Applicant: BIOGATE PRECISION MEDICINE CORP., Taipei City (TW)

(72) Inventors: Chong-Jen Frank HUANG, Taipei City (TW); Chia-Wen HO, Taipei City (TW)

(21) Appl. No.: 19/295,719

(22) Filed: Aug. 11, 2025

Related U.S. Application Data
(63) Continuation-in-part of application No. 18/846,269.
(60) Provisional application No. 63/430,075, filed on Dec. 5, 2022.

(51) Int. Cl. C07K 16/26; A61K 39/00; A61P 3/00; A61P 35/00

(52) U.S. Cl. CPC C07K 16/26; A61P 3/00; A61P 35/00; A61K 2039/505; C07K 2317/24; C07K 2317/565; C07K 2317/73; C07K 2317/76; C07K 2317/92

(57) ABSTRACT

Disclosed herein is a method of treating cachexia in a subject. According to some embodiments of the present disclosure, the method includes administering to the subject an effective amount of a recombinant antibody or a fragment thereof, which exhibits a binding affinity and a neutralizing effect on parathyroid hormone-related protein. According to certain embodiments of the present disclosure, the cachexia is caused by a parathyroid hormone-related protein (PTHrP)-expressing cancer, chronic kidney disease, kidney failure, heart failure, or tuberculosis.

### CROSS-REFERENCE TO RELATED APPLICATIONS

This application is a continuation-in-part of U.S. application Ser. No. 18/846,269 filed Sep. 12, 2024, which is a national application of PCT/US2023/082587 filed Dec. 5, 2023 and claims the benefit of U.S. Provisional Application No. 63/430,075, filed Dec. 5, 2022; the content of the applications is incorporated herein by reference in its entirety.

### REFERENCES TO A SEQUENCE LISTING

The present application is being filed along with a Sequence Listing in an electronic format. The Sequence Listing is provided as a file entitled “P4304-1_SEQ_AF.xml”, created Aug. 10, 2025, which is 19,243 bytes in size. The information in the electronic format of the Sequence Listing is incorporated herein by reference in its entirety.

### BACKGROUND OF THE INVENTION

### 1. Field of the Invention

The present disclosure in general relates to the field of disease treatment. More particularly, the present disclosure relates to methods of treating cachexia by using a recombinant antibody specific to parathyroid hormone-related protein (PTHrP).

### 2. Description of Related Art

Cachexia, also known as “wasting syndrome”, is a metabolic disorder that comes with the advanced-stage or end-stage of several diseases and illnesses, including chronic kidney disease (CKD), kidney failure, heart failure, infectious disease (e.g., acquired immunodeficiency syndrome (AIDS)), multiple sclerosis, tuberculosis, rheumatoid arthritis, cystic fibrosis, chronic obstructive pulmonary disease (COPD), and cancer (i.e., cancer cachexia, CC), and is characterized by body weight loss and muscular atrophy and adipose tissue depletion, or without any association with another diseases, illnesses, or medical conditions. It affects approximately 70%-80% of cancer patients and is responsible for up to 22%-30% of cancer deaths depending on the type of cancer. Several factors are involved in the development and/or progression of cancer cachexia, including anorexia, skeletal muscle breakdown, and various metabolic alterations in cancer patients. In general, cancer cells cause the immune system to release inflammatory cytokines (e.g., interleukin (IL)-1, IL-6, IL-8, and tumor necrosis factor-alpha (TNF-α)) that lead to muscle and fat loss, as well as speed up the metabolic rate of the cancer patients. The presence of cachexia in cancer patients is associated with decline in physical function, diminished quality of life, tolerance to anticancer therapies, and poor survival rates.

Cachexia is neither preventable nor reversible. No widely approved drug for the treatment of cachexia is available. Current treatment for cachexia has been focused on the supplement of nutrition (e.g., branched-chain amino acids or fatty acids), the administration of appetite stimulant (e.g., megestrol or dronabinol), the administration of corticosteroids (e.g., prednisolone, methylprednisolone, and dexamethasone), the administration of non-steroidal anti-inflammation drug (e.g., nonsteroidal anti-inflammatory drugs (NSAIDs) or steroids), exercise, and psychosocial interventions. Unfortunately, none of the treatments provides a satisfactory effect on cachectic patients. For example, the supplement of nutrition alone is insufficient to stop cachexia. Its benefits are observed only when taken with other treatments. The appetite stimulant and anti-inflammatory drug may cause various side effects such as bleeding, insulin resistance, nephrotoxicity, venous thromboembolism, hypogonadism, adrenal insufficiency, edema, and increased mortality in older patients. Also, side effects and serious complications (e.g., fatigue, anxiety, hearing loss, abdominal pain or cramps, diarrhoea, nausea, and cardiac arrhythmias) associated with the gastrointestinal hypermotility drugs have been reported. Exercise may attenuate cachexia progression, but the evidence is currently not sufficient to recommend it as cachexia treatment. Further, exercise is not recommended for patients with frailty, sarcopenia, or other acute illnesses. Regarding the psychosocial interventions, their effectiveness still needs to be further verified.

In view of the foregoing, there is a continuing interest in developing a novel method and/or agent for treating cachexia in a safer and more efficient manner.

### SUMMARY

The following presents a simplified summary of the disclosure in order to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later.

The present disclosure provides a method of treating cachexia, including cancer cachexia (i.e., cachexia caused by and/or associated with cancer) in a subject. According to the embodiments of the present disclosure, the method comprises administering to the subject an effective amount of a recombinant antibody. The recombinant antibody comprises in its structure, a heavy chain variable (VH) domain and a light chain variable (VL) domain, in which the VH domain comprises a first heavy chain complementarity determining region (CDR-H1), a second heavy chain CDR (CDR-H2), and a third heavy chain CDR (CDR-H3); and the VL domain comprises a first light chain CDR (CDR-L1), a second light chain CDR (CDR-L2), and a third light chain CDR (CDR-L3).

According to some embodiments of the present disclosure, the CDR-H1, CDR-H2 and CDR-H3 respectively comprise the amino acid sequences of “GYXFTDY” (SEQ ID NO: 1), “DTSDSY” (SEQ ID NO: 2), and “GDY”, in which the “X” residue at position 3 of SEQ ID NO: 1 is isoleucine (I) or threonine (T). In the embodiments, the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4).

According to certain embodiments of the present disclosure, the recombinant antibody is designated as 16G2, in which the CDR-H1, CDR-H2 and CDR-H3 respectively comprise the amino acid sequences of “GYIFTDY” (SEQ ID NO: 5), “DTSDSY” (SEQ ID NO: 2), and “GDY”; and the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4).

Depending on desired purpose, the antibody 16G2 may be produced in the form of a murine antibody (i.e., both variable and constant domains of the antibody are derived from mouse), a chimeric antibody (i.e., variable and constant domains of the antibody are respectively derived from mouse and human) or a humanized antibody (i.e., the framework sequences of variable domains of the antibody are modified in accordance with antibody variants produced naturally in human).

According to one embodiment, the antibody 16G2 is produced in the form of a murine antibody, in which the VH and VL domains respectively comprise the amino acid sequences at least 85% identical to SEQ ID NOs: 7 and 8. In one exemplary embodiment, the VH and VL domains of the murine antibody 16G2 respectively comprise the amino acid sequences 100% identical to SEQ ID NOs: 7 and 8, i.e., the VH and VL domains of the murine antibody 16G2 respectively comprise the amino acid sequences of SEQ ID NOs: 7 and 8.

According to another embodiment, the antibody 16G2 is produced in the form of a chimeric antibody, in which the VH and VL domains respectively comprise the amino acid sequences at least 85% identical to SEQ ID NOs: 7 and 8; preferably, the VH and VL domains of the chimeric antibody 16G2 respectively comprise the amino acid sequences of SEQ ID NOs: 7 and 8. In this embodiment, the chimeric antibody 16G2 further comprises a heavy chain constant (CH) and a light chain constant (CL) domains derived from a human immunoglobulin, in which the CH and CL domains respectively comprise the amino acid sequences at least 85% identical to SEQ ID NOs: 18 and 19; preferably, the CH and CL domains of the chimeric antibody 16G2 respectively comprise the amino acid sequences of SEQ ID NOs: 18 and 19.

According to still another embodiment, the antibody 16G2 is produced in the form of a humanized antibody, in which the VH domain comprises the amino acid sequence at least 85% identical to SEQ ID NOs: 9, 10, 11 or 12; and the VL domain comprises the amino acid sequence at least 85% identical to SEQ ID NOs: 13, 14 or 15. In one exemplary embodiment, the VH domain of the humanized antibody 16G2 comprises the amino acid sequence of SEQ ID NOs: 9, 10, 11 or 12; and the VL domain of the humanized antibody 16G2 comprises the amino acid sequence of SEQ ID NOs: 13, 14 or 15. In one specific embodiment, the VH domain of the humanize antibody 16G2 comprises the amino acid sequence of SEQ ID NO: 11; and the VL domain of the humanize antibody 16G2 comprises the amino acid sequence of SEQ ID NO: 13.

According to some embodiments of the present disclosure, the recombinant antibody is designated as 5B3, in which the CDR-H1, CDR-H2 and CDR-H3 respectively comprise the amino acid sequences of “GYTFTDY” (SEQ ID NO: 6), “DTSDSY” (SEQ ID NO: 2), and “GDY”; and the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4). As could be appreciated, the antibody 5B3 may be produced in the form of a murine antibody, a chimeric antibody or a humanized antibody in accordance with desired purpose.

According to one embodiment, the antibody 5B3 is produced in the form of a murine antibody, in which the VH and VL domains respectively comprise the amino acid sequences at least 85% identical to SEQ ID NOs: 16 and 17. In one exemplary embodiment, the VH and VL domains of the murine antibody 5B3 respectively comprise the amino acid sequences of SEQ ID NOs: 16 and 17.

According to another embodiment, the antibody 5B3 is produced in the form of a chimeric antibody, in which the VH and VL domains respectively comprise the amino acid sequences at least 85% identical to SEQ ID NOs: 16 and 17; preferably, the VH and VL domains of the chimeric antibody 5B3 respectively comprise the amino acid sequences of SEQ ID NOs: 16 and 17. In this embodiment, the chimeric antibody 5B3 further comprises a CH and a CL domains derived from a human immunoglobulin, wherein the CH and CL domains respectively comprise the amino acid sequences at least 85% identical to SEQ ID NOs: 18 and 19; preferably, the CH and CL domains of the chimeric antibody 5B3 respectively comprise the amino acid sequences of SEQ ID NOs: 18 and 19.

According to various embodiments, the cachexia is caused by and/or associated with cancer, CKD, kidney failure, heart failure, infectious disease, multiple sclerosis, tuberculosis, rheumatoid arthritis, cystic fibrosis, COPD, or a combination thereof. As its name implies, the cancer cachexia is caused by and/or associated with a cancer, which preferably is a PTHrP-expressing cancer (i.e., the cancer expressing or secreting PTHrP); for example, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

In another exemplary embodiment, the cachexia is caused by and/or associated with CKD or kidney failure.

Also disclosed therein are the uses of the recombinant antibody of the present disclosure in the preparation of a medicament or a pharmaceutical composition for treating cachexia in a subject. The medicament or pharmaceutical composition comprises the present recombinant antibody and, optionally, a pharmaceutically acceptable carrier.

The subject treatable with the present recombinant antibody, medicament, pharmaceutical composition and/or method is a mammal; preferably, a human.

Many of the features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1 depicts the effect of the recombinant antibody BGM-2121 on tumor growth and phosphokinase (CPK) of mice receiving specified treatment according to Example 2 of the present disclosure. (A) tumor weight of BxPC-3 liver metastasis mice with indicated treatment; (B) CPK level of BxPC-3 liver metastasis mice with indicated treatment.

FIG. 2A depicts the effect of the recombinant antibody BGM-2121 on tumor volume of mice receiving specified treatment according to Example 3.1 of the present disclosure.

FIG. 2B depicts the effect of the recombinant antibody BGM-2121 on body weight of mice receiving specified treatment according to Example 3.1 of the present disclosure.

FIG. 2C depicts the effect of the recombinant antibody BGM-2121 on tumor weight of HARA-B-bearing mice receiving specified treatment according to Example 3.1 of the present disclosure.

FIG. 2D depicts the effect of the recombinant antibody BGM-2121 on carcass weight of HARA-B-bearing mice receiving specified treatment according to Example 3.1 of the present disclosure.

FIG. 2E depicts the effect of the recombinant antibody BGM-2121 on body mass of mice receiving specified treatment according to Example 3.1 of the present disclosure, in which the assessment of body composition was made through whole-body microCT imaging, * p<0.05; *** p<0.001, student's t test.

FIG. 2F depicts the effect of the recombinant antibody BGM-2121 on lean body mass of mice receiving specified treatment according to Example 3.1 of the present disclosure, in which the assessment of body composition was made through whole-body microCT imaging, * p<0.05; *** p<0.001, student's t test.

FIG. 2G depicts the effect of the recombinant antibody BGM-2121 on adipose mass of HARA-B-bearing mice with indicated treatment according to Example 3.1 of the present disclosure, in which the assessment of body composition was made through whole-body microCT imaging, * p<0.05 ** p<0.01.

FIG. 2H depicts the effect of the recombinant antibody BGM-2121 on skeletal muscle fiber cross-section area of quadriceps femoris (QF), gastrocnemius (GA), tibialis anterior (TA), and soleus (SOL) of HARA-B-bearing mice with indicated treatment according to Example 3.1 of the present disclosure, * p<0.05 ** p<0.01.

FIG. 3 depicts the effect of the recombinant antibody BGM-2121 on (A) serum level of PTHrP and (B) calcium level of HARA-B-bearing mice receiving specified treatment according to Example 3.1 of the present disclosure, *** P<0.001 student's t test.

FIG. 4A depicts the effect of the recombinant antibody BGM-2121 on tumor volume of 786-O-bearing mice receiving specified treatment according to Example 3.2 of the present disclosure.

FIG. 4B depicts the effect of the recombinant antibody BGM-2121 on body weight of 786-O-bearing mice receiving specified treatment according to Example 3.2 of the present disclosure.

FIG. 4C depicts the effect of the recombinant antibody BGM-2121 on tumor weight of 786-O-bearing mice receiving specified treatment according to Example 3.2 of the present disclosure.

FIG. 4D depicts the effect of the recombinant antibody BGM-2121 on carcass weight of 786-O-bearing mice receiving specified treatment according to Example 3.2 of the present disclosure.

FIG. 4E depicts the effect of the recombinant antibody BGM-2121 on body mass of 786-O-bearing mice receiving specified treatment according to Example 3.2 of the present disclosure, in which the assessment of body composition was made through whole-body microCT imaging, * p<0.05; ** p<0.01; *** p<0.001, student's t test.

FIG. 4F depicts the effect of the recombinant antibody BGM-2121 on lean body mass of 786-O-bearing mice receiving specified treatment according to Example 3.2 of the present disclosure, in which the assessment of body composition was made through whole-body microCT imaging, * p<0.05; ** p<0.01; *** p<0.001, student's t test.

FIG. 4G depicts the effect of the recombinant antibody BGM-2121 on adipose mass of 786-O-bearing mice receiving specified treatment according to Example 3.2 of the present disclosure, in which the assessment of body composition was made through whole-body microCT imaging, * p<0.05; ** p<0.01; *** p<0.001, student's t test.

FIG. 4H depicts the effect of the recombinant antibody BGM-2121 on muscle fiber cross-section area of quadriceps femoris (QF), gastrocnemius (GA), tibialis anterior (TA), and soleus (SOL) of 786-O-bearing mice receiving specified treatment according to Example 3.2 of the present disclosure, * p<0.05 vs. Healthy control; #p<0.05 vs. hIgG group.

FIG. 4I depicts the effect of the recombinant antibody BGM-2121 on adipocyte tissue size of 786-O-bearing mice receiving specified treatment according to Example 3.2 of the present disclosure, including the sizes of inguinal white adipose tissue (iWAT), epididymal white adipose tissue (eWAT), and interscapular brown adipose tissue (iBAT). * p<0.05 vs. Healthy control; #p<0.05 vs. hIgG group.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “cancer cachexia”, also known as “cancer anorexia cachexia”, shall be understood to refer to cachexia that is associated with cancer or occurring in a subject that is suffering from cancer. Cancer cachexia is characterized by an ongoing loss of muscle mass (with or without loss of fat mass), leading to progressive functional impairment which cannot be fully reversed by normal nutritional support.

The term “antibody” (Ab) is used in the broadest sense and specifically covers monoclonal antibodies (including full length monoclonal antibodies), polyclonal antibodies, multi-specific or multivalent antibodies (e.g., bi-specific antibodies), chimeric antibodies, humanized antibodies and antibody fragments so long as they exhibit the desired biological activity. The term “antibody fragment” or “the fragment of an antibody” refers to a portion of a full-length antibody, generally the antigen binding or variable domain (i.e., VH and VL domains) of a full-length antibody. Examples of the antibody fragment include fragment antigen-binding (Fab), Fab′, F(ab′)2, single-chain variable fragment (scFv), diabody, linear antibody, single-chain antibody molecule, and multi-specific antibody formed from antibody fragments.

The term “complementarity determining region” (CDR) used herein refers to the hypervariable region of an antibody molecule that forms a surface complementary to the three-dimensional surface of a bound antigen. Proceeding from N-terminus to C-terminus, each of the antibody heavy and light chains comprises three CDRs (CDR-1, CDR-2 and CDR-3). An antigen combining site, therefore, includes a total of six CDRs that comprise three CDRs in the variable domain of a heavy chain (i.e., CDR-H1, CDR-H2 and CDR-H3), and three CDRs in the variable domain of a light chain (i.e., CDR-L1, CDR-L2 and CDR-L3).

The “variable domain” of an antibody refers to the amino-terminal domains of heavy or light chain of the antibody. These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, Fifth Edition, National Institute of Health, Bethesda, Md. (1991)).

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. The antibody of the present disclosure may be modified specifically to alter a feature of the antibody unrelated to its physiological activity. For example, certain amino acid residues in the framework (FR) region of the antibody can be changed and/or deleted without affecting the physiological activity of the antibody in this study (i.e., its ability to treat cancer cachexia). In particular, conservative amino acid replacements are contemplated. Conservative replacements are those that take place within a family of amino acid residues that are related in their side chains. Genetically encoded amino acid residues are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid residue within the antigen-biding sites, i.e., CDRs. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains.

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software.

Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

As used herein, the term “treat”, “treating” and “treatment” are interchangeable and encompasses partially or completely preventing, ameliorating, mitigating and/or managing a symptom, a secondary disorder or a condition associated with cachexia. The term “treating” as used herein refers to application or administration of the recombinant antibody of the present disclosure to a subject, who has a symptom, a secondary disorder or a condition associated with cachexia, with the purpose to partially or completely alleviate, ameliorate, relieve, delay onset of, inhibit progression of, reduce severity of, and/or reduce incidence of one or more symptoms, secondary disorders or features associated with cachexia. Symptoms, secondary disorders, and/or conditions associated with cachexia include, but are not limited to, asthenia, anorexia, early satiety, nausea, taste change, loss of appetite, weight loss (including loss of fat and/or muscle mass), anemia, fatigue, weakness, and hormonal aberration. Treatment may be administered to a subject who exhibits only early signs of such symptoms, disorder, and/or condition for the purpose of decreasing the risk of developing the symptoms, secondary disorders, and/or conditions associated with cachexia. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered, or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the recombinant antibody of the present disclosure), such as molar concentration, mass concentration, volume concentration, molality, mole fraction, mass fraction and mixing ratio. Persons having ordinary skills could calculate the human equivalent dose (HED) for the medicament (such as the present recombinant antibody) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the recombinant antibody, medicament, pharmaceutical composition and/or method of the present invention. The term “subject” is intended to refer to both the male and female gender unless one gender is specifically indicated.

### II. Description of the Invention

### (i) Antibodies-Murine mAb 16G2 and Murine mAb 5B3

The first aspect of present disclosure is directed to two monoclonal antibodies (mAbs) respectively designated as “16G2” and “5B3”. According to certain embodiments of the present disclosure, each of the present mAbs exhibits a binding affinity to and a neutralizing effect on PTHrP, an endocrine, autocrine, paracrine and intracrine hormone; and the administration of the present mAb is capable of treating cachexia caused by and/or associated with a PTHrP-related cancer (i.e., PTHrP-related cancer cachexia).

According to some embodiments of the present disclosure, the present mAb is produced by immunization method (i.e., immunizing animals with specific peptides).

In general, the polypeptide (i.e., PTHrP polypeptide) can be synthesized by commonly used methods such as t-BOC or FMOC protection of alpha-amino groups. Both methods involve stepwise syntheses whereby a single amino acid is added at each step starting from the C terminus of the peptide. Polypeptides of the invention can also be synthesized by the well-known solid phase peptide synthesis methods.

Then, the antibody can be produced by immunizing a host animal, such as a mouse, a rat, or a rabbit, with the synthetic polypeptide. The immunization may be performed in accordance with commonly adopted procedures. The immunization interval is not particularly limited. Immunization may be carried out at intervals of several days to several weeks, preferably one week, for 2-10 times, until a desired antibody titer is reached. For example, the host animals may be vaccinated by subcutaneously injecting with the synthetic polypeptide on weekly basis for 8 consecutive weeks.

After the final immunization, splenic cells and regional lymph nodes are removed. Blood samples are taken regularly after immunization and subject to centrifugation to separate sera. The resultant sera are then subject to measurement of antibody titers by any suitable method, which includes, but is not limited to, enzyme linked immunosorbent assay (ELISA), enzyme immunoassay (EIA), or radio immunoassay (RIA). In one preferred example, antibody titers are measured by ELISA. Then, final immunization is given to those animals showing high antibody titers to the synthetic polypeptide. Antibody-producing cells are prepared from splenic cells and regional lymph nodes or the like of the immunized animals. In the preparation of antibody-producing cells, it is preferable to remove tissue debris and erythrocytes as much as possible. Commercial erythrocyte remover may be used to this purpose. Alternatively, a buffer ammonium chloride and Tris may be prepared and used. The thus prepared antibody-producing cells should be immediately fused with immortal cells such as myeloma cells to produce hybridoma cells, which semi-eternally continue to proliferate while producing antibodies. Commonly available cell strains derived from an animal such as mouse may be used. A preferable cell strain to be used in this invention should not survive in HAT selection medium, which contains hypoxanthine, thymidine and aminopterin; and should survive there only when fused with antibody-producing cells. Examples of myeloma cells include, but are not limited to, mouse myeloma cell line (such as myeloma FO cells) and human myeloma cell line (such as Karpas 707H). Cell fusion is usually carried out by mixing splenic cells or lymph node cells with commercially available myeloma cells in the presence of a cell-fusion promoter, such as polyethylene glycol (PEG) having an average molecular weight from about 200 to 20,000 daltons or the like. Alternatively, cell fusion may be carried out in a commercial cell fusion device utilizing electric stimulation such as electroporation. After the fusion, the resultant cells are then diluted and cultured in HAT medium.

Hybridomas of interest are then selected from the fused cells. The fused cells cultured and survived in HAT medium would form colonies. The supernatant of each culture well is then collected and examined for the presence or absence of antibody titers to the polypeptide. As a method of confirmation, ELISA, EIA or RIA may be used. Once antibody-positive wells are identified, cells are then cultured in a HT medium, which merely contains hypoxanthine and thymidine, without aminopterin. After culturing for a while, antibody titers in the culture supernatant are confirmed again. Cells that are finally selected are then subject to cloning to obtain single cells. Clones that exhibit high specificity to the polypeptide are selected and proliferated to some extent to establish hybridomas.

The mAbs produced by the hybridomas may be isolated or prepared by any known method. For example, antibodies may be prepared from cultured supernatant obtained by culturing hybridomas in a medium with low serum concentration. Alternatively, hybridomas may be injected into abdominal cavities of animals and the resultant abdominal dropsies are collected to prepare antibodies. Antibodies may be purified or isolated by methods that employ affinity column, gel filtration chromatography, ion exchange chromatography or the like. Any of these known methods may be appropriately selected or used in combination.

In structure, each of the thus-produced mAbs 16G2 and 5B3 comprises three CDRs in the VH domain thereof (i.e., CDR-H1, CDR-H2, and CDR-H3), and three CDRs in the VL domain thereof (i.e., CDR-L1, CDR-L2, and CDR-L3).

According to some embodiments of the present disclosure, the CDR-H1, CDR-H2, and CDR-H3 of mAb 16G2 respectively comprise the amino acid sequences of “GYIFTDY” (SEQ ID NO: 5), “DTSDSY” (SEQ ID NO: 2), and “GDY”; and the CDR-L1, CDR-L2, and CDR-L3 of mAb 16G2 respectively comprise the amino acid sequences of “QSLLESDGKTY” (SEQ ID NO: 3), “LVS”, and “CQGTHFPWT” (SEQ ID NO: 4).

As an example, the amino acid sequences of the VH and VL domains of mAb 16G2 are respectively provided as SEQ ID NOs: 7 and 8, described below, in which the CDRs (i.e., the CDR-H1, CDR-H2 and CDR-H3 of VH domain, and the CDR-L1, CDR-L2 and CDR-L3 of VL domain) are marked in bold, in sequence.

(VH domain of mAb 16G2)
SEQ ID NO: 7
QVQLQQPGAELGMPGASVKMSCKASGYIFTDY
WMHWVKQRPGQGLEWIGAIDTSDSYSSYNQKF
QGKATLTVDESSSTAYMQLSSLTSEDSAVYYC
TLGDYWGQGTTLTVSS
(VL domain of mAb 16G2)
SEQ ID NO: 8
DVVMTQTPLTLSVTIGQPASMSCKSSQSLLESDGKTY
LNWLLQRPGQSPKRLIYLVSKLDSGVPDRFTGSGSGT
DFTLKISRVEAEDLGVYYCCQGTHFPWTFGGGTKLEI
K

Since the binding affinity and specificity of an antibody are mainly determined by the CDR sequences thereof, as could be appreciated, the framework (FR) sequences of the VH and VL domains may vary (e.g., being substituted by conserved or non-conserved amino acid residues) without affecting the binding affinity and/or specificity of the present antibody. Preferably, the FR sequence is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue).

Based on the conservative substitution, a skilled artisan may substitute the amino acid residue(s) of the FR sequences of the VH and VL domains of mAb 16G2 without affecting the activity and/or effect of mAb 16G2 (i.e., neutralizing PTHrP and treating cachexia). Accordingly, the antibody comprising substituted amino acid(s) in its FR sequences of VH and VL domains are intended to be included within the scope of the present disclosure. According to certain embodiments, the VH domain of mAb 16G2 comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 7, and the VL domain of mAb 16G2 comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 8. According to some preferred embodiments, the VH and VL domains of mAb 16G2 respectively comprise the amino acid sequences at least 90% identical to SEQ ID NOs: 7 and 8. More preferably, the VH and VL domains of mAb 16G2 respectively comprise the amino acid sequences at least 95% identical to SEQ ID NOs: 7 and 8.

According to some embodiments of the present disclosure, the CDR-H1, CDR-H2, and CDR-H3 of mAb 5B3 respectively comprise the amino acid sequences of “GYTFTDY” (SEQ ID NO: 6), “DTSDSY” (SEQ ID NO: 2), and “GDY”; and the CDR-L1, CDR-L2, and CDR-L3 of mAb 5B3 respectively comprise the amino acid sequences of “QSLLESDGKTY” (SEQ ID NO: 3), “LVS”, and “CQGTHFPWT” (SEQ ID NO: 4).

As an example, the amino acid sequences of the VH and VL domains of mAb 5B3 are respectively provided as SEQ ID NOs: 16 and 17, described below, in which the CDRs (i.e., the CDR-H1, CDR-H2 and CDR-H3 of VH domain, and the CDR-L1, CDR-L2 and CDR-L3 of VL domain) are marked in bold, in sequence.

(VH domain of mAb 5B3)
SEQ ID NO: 16
QVQLQQPGAELVMPGASVKMSCKASGYTFTDY
WMHWVKQRPGQGLEWIGALDTSDSYASYNQRF
KGKATLTVDASSSTAYMQLSSLTSEDSAVYYC
TLGDYWGQGTTLTVSS
(VL domain of mAb 5B3)
SEQ ID NO: 17
DVVMTQTPLTLSVTFGQPASISCKSSQSLLESDGKTY
LNWLLQRPGQSPKRLIYLVSKLDSGVPDRFTGSGSGT
DFTLKISRVEAEDLGVYYCCQGTHFPWTFGGGTKLEI
K

As described above, the FR sequences of the VH and VL domains may vary (e.g., being substituted by conserved or non-conserved amino acid residues) without affecting the binding affinity and/or specificity of the present antibody, and a skilled artisan may substitute the amino acid residue(s) of the FR sequences of the VH and VL domains of mAb 5B3 without affecting the activity and/or effect of mAb 5B3 (i.e., neutralizing PTHrP and treating cachexia). Accordingly, the antibody comprising substituted amino acid(s) in its FR sequences of VH and VL domains are intended to be included within the scope of the present disclosure. According to certain embodiments, the VH domain of mAb 5B3 comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 16, and the VL domain of mAb 5B3 comprises the amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 17. According to some preferred embodiments, the VH and VL domains of mAb 5B3 respectively comprise the amino acid sequences at least 90% identical to SEQ ID NOs: 16 and 17. More preferably, the VH and VL domains of mAb 5B3 respectively comprise the amino acid sequences at least 95% identical to SEQ ID NOs: 16 and 17.

As could be appreciated, the present mAb (i.e., mAb 16G2 or 5B3) may alternatively be produced by DNA cloning based the amino acid sequences disclosed above. Specifically, a skilled artisan may produce a DNA construct that comprises the nucleotide sequences encoding the VH and VL sequences of the present mAb, followed by transfecting the DNA construct into suitable host cells, such as E. Coli cells, simian COS cells, Chinese hamster ovary (CHO) cells or myeloma cells that do not produce immunoglobulin proteins, to synthesize the desired mAb in the recombinant host cells.

Depending on intended purpose, the present mAb may be produced in the form of an immunoglobulin G (IgG), immunoglobulin A (IgA), immunoglobulin M (IgM), immunoglobulin D (IgD) or immunoglobulin E (IgE).

### (ii) Antibodies-Chimeric mAb 16G2 and Chimeric mAb 5B3

Each of the mAbs (including mAbs 16G2 and 5B3) described in Section (i) of the present disclosure is useful in producing a chimeric antibody, i.e., the antibody having variable domains from its original species (e.g., a mouse) and constant domains from another species (e.g., a human), so as to reduce the immunogenicity of the antibody in a subject.

Accordingly, the present disclosure also provides two chimeric antibodies respectively derived from murine mAbs 16G2 and 5B3, in which the constant domains of the murine mAbs are replaced with those of a human antibody.

According to certain embodiments, the VH domain of chimeric mAb 16G2 comprises the amino acid sequence of SEQ ID NO: 7 as described in Section (i) of the present disclosure, and the VL domain of chimeric mAb 16G2 comprises the amino acid sequence of SEQ ID NO: 8.

According to some embodiments, the VH domain of chimeric mAb 5B3 comprises the amino acid sequence of SEQ ID NO: 16 as described in Section (i) of the present disclosure, and the VL domain of chimeric mAb 5B3 comprises the amino acid sequence of SEQ ID NO: 17.

In these embodiments, each of the chimeric mAbs 16G2 and 5B3 comprises a CH domain of SEQ ID NO: 18 linked to its VH domain, and a CL domain of SEQ ID NO: 19 linked to its VL domain. The amino acid sequences of the CH and CL are provided below.

(CH domain of chimeric mAbs)
SEQ ID NO: 18
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(CL domain of chimeric mAbs)
SEQ ID NO: 19
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

As described, the constant domains and the FR sequences of the VH and VL domains may vary (e.g., being substituted by conserved or non-conserved amino acid residues) without affecting the binding affinity and/or specificity of the present antibody, and a skilled artisan may substitute the amino acid residue(s) of the constant domains and/or the FR sequences of the VH and VL domains of present chimeric mAb without affecting its activity and/or effect (i.e., neutralizing PTHrP and treating cancer cachexia). Accordingly, the antibody comprising substituted amino acid(s) in its constant domains and/or FR sequences of VH and VL domains are intended to be included within the scope of the present disclosure.

Depending on intended purpose, the chimeric mAb may be produced in the form of an IgG, IgA, IgM, IgD or IgE.

(iii) Antibodies-Humanized mAb 16G2

Alternatively, each of the mAbs (including mAbs 16G2 and 5B3) described in Section (i) of the present disclosure is useful in producing a humanized antibody, i.e., the amino acid sequences of a non-human antibody (e.g., a murine antibody) are modified to increase its similarity to the antibody variant produced naturally in humans, so as to minimize the immunogenicity of the antibody in a human subject.

Accordingly, the present disclosure further provides different humanized VH and VL sequences, including humanized VH1 (SEQ ID NO: 9), humanized VH2 (SEQ ID NO: 10), humanized VH3 (SEQ ID NO: 11), humanized VH4 (SEQ ID NO: 12), humanized VL1 (SEQ ID NO: 13), humanized VL2 (SEQ ID NO: 14), and humanized VL3 (SEQ ID NO: 15). The amino acid sequences of the humanized VH and VL domains are provided below, in which the CDRs (i.e., the CDR-H1, CDR-H2 and CDR-H3 of VH domain, and the CDR-L1, CDR-L2 and CDR-L3 of VL domain) are marked in bold, in sequence.

(humanized VH1 domain of mAb 16G2)
SEQ ID NO: 9
QVQLVQSGAEVKKPGASVKVSCKASGYIFTDY
WMHWVRQAPGQGLEWMGAIDTSDSYSSYNQKF
QGRVTMTRDTSTSTVYMELSSLRSEDTAVYYC
TLGDYWGQGTTVTVSS
(humanized VH2 domain of mAb 16G2)
SEQ ID NO: 10
QVQLVQSGAEVKKPGASVKVSCKASGYIFTDY
WMHWVRQAPGQGLEWIGAIDTSDSYSSYNQKF
QGRATMTVDTSTSTVYMELSSLRSEDTAVYYC
TLGDYWGQGTTVTVSS
(humanized VH3 domain of mAb 16G2)
SEQ ID NO: 11
QVQLVQSGAEVKKPGASVKVSCKASGYIFTDY
WMHWVKQAPGQGLEWIGAIDTSDSYSSYNQKF
QGRATMTVDTSTSTVYMELSSLRSEDTAVYYC
TLGDYWGQGTTVTVSS
(humanized VH4 domain of mAb 16G2)
SEQ ID NO: 12
QVQLVQSGAEVKKPGASVKVSCKASGYIFTDY
WMHWVKQAPGQGLEWIGAIDTSDSYSSYNQKF
QGRATMTVDESTSTVYMELSSLRSEDTAVYYC
TLGDYWGQGTTVTVSS
(humanized VL1 domain of mAb 16G2)
SEQ ID NO: 13
DVVMTQSPLSLPVTLGQPASISCKSSQSLLES
DGKTYLNWFQQRPGQSPRRLIYLVSKLDSGVP
DRFSGSGSGTDFTLKISRVEAEDVGVYYCCQG
THFPWTFGGGTKLEIK
(humanized VL2 domain of mAb 16G2)
SEQ ID NO: 14
DVVMTQSPLSLPVTLGQPASISCKSSQSLLES
DGKTYLNWLLQRPGQSPRRLIYLVSKLDSGVP
DRFSGSGSGTDFTLKISRVEAEDVGVYYCCQG
THFPWTFGGGTKLEIK
(humanized VL3 domain of mAb 16G2)
SEQ ID NO: 15
DVVMTQSPLSLPVTLGQPASMSCKSSQSLLES
DGKTYLNWLLQRPGQSPKRLIYLVSKLDSGVP
DRFSGSGSGTDFTLKISRVEAEDVGVYYCCQG
THFPWTFGGGTKLEIK

As described, the FR sequences of the VH and VL domains may vary (e.g., being substituted by conserved or non-conserved amino acid residues) without affecting the binding affinity and/or specificity of the present antibody, and a skilled artisan may substitute the amino acid residue(s) of the FR sequences of the VH and VL domains of present humanized mAb without affecting its activity and/or effect (i.e., neutralizing PTHrP and treating cachexia). Accordingly, the antibody comprising substituted amino acid(s) in the FR sequences of the humanized VH and VL domains are intended to be included within the scope of the present disclosure.

According to one embodiment, the humanized mAb designated as “humanized mAb 16G2-1” comprises the VH1 domain (SEQ ID NO: 9) and VL1 domain (SEQ ID NO: 13). According to another embodiment, the humanized mAb designated as “humanized mAb 16G2-2” comprises the VH1 domain (SEQ ID NO: 9) and VL2 domain (SEQ ID NO: 14). According to another embodiment, the humanized mAb designated as “humanized mAb 16G2-3” comprises the VH1 domain (SEQ ID NO: 9) and VL3 domain (SEQ ID NO: 15).

According to one embodiment, the humanized mAb designated as “humanized mAb 16G2-4” comprises the VH2 domain (SEQ ID NO: 10) and VL1 domain (SEQ ID NO: 13). According to another embodiment, the humanized mAb designated as “humanized mAb 16G2-5” comprises the VH2 domain (SEQ ID NO: 10) and VL2 domain (SEQ ID NO: 14). According to another embodiment, the humanized mAb designated as “humanized mAb 16G2-6” comprises the VH2 domain (SEQ ID NO: 10) and VL3 domain (SEQ ID NO: 15).

According to one embodiment, the humanized mAb designated as “humanized mAb 16G2-7” comprises the VH3 domain (SEQ ID NO: 11) and VL1 domain (SEQ ID NO: 13). According to another embodiment, the humanized mAb designated as “humanized mAb 16G2-8” comprises the VH3 domain (SEQ ID NO: 11) and VL2 domain (SEQ ID NO: 14). According to another embodiment, the humanized mAb designated as “humanized mAb 16G2-9” comprises the VH3 domain (SEQ ID NO: 11) and VL3 domain (SEQ ID NO: 15).

According to one embodiment, the humanized mAb designated as “humanized mAb 16G2-10” comprises the VH4 domain (SEQ ID NO: 12) and VL1 domain (SEQ ID NO: 13). According to another embodiment, the humanized mAb designated as “humanized mAb 16G2-11” comprises the VH4 domain (SEQ ID NO: 12) and VL2 domain (SEQ ID NO: 14). According to another embodiment, the humanized mAb designated as “humanized mAb 16G2-12” comprises the VH4 domain (SEQ ID NO: 12) and VL3 domain (SEQ ID NO: 15).

Depending on intended purpose, the humanized mAb may be produced in the form of an IgG, IgA, IgM, IgD or IgE.

According to some embodiments of the present disclosure, the present mAb (including the murine mAb, chimeric mAb and humanized mAb respectively described in Sections (i)-(iii) of the present disclosure) exhibits a binding affinity and neutralizing activity toward PTHrP.

### (iv) Medicament or Pharmaceutical Composition Comprising the Present mAb

Another aspect of the present disclosure pertains to a medicament or a pharmaceutical composition for the treatment of cachexia (e.g., cancer cachexia). The medicament or pharmaceutical composition comprises the mAb of the present disclosure, and optionally, a pharmaceutically acceptable carrier.

Generally, the mAb of this invention is present at a level of about 0.1% to 99% by weight, based on the total weight of the medicament or a pharmaceutical composition. In some embodiments, the mAb of this invention is present at a level of at least 1% by weight, based on the total weight of the medicament or a pharmaceutical composition. In certain embodiments, the mAb is present at a level of at least 5% by weight, based on the total weight of the medicament or a pharmaceutical composition. In still other embodiments, the mAb is present at a level of at least 10% by weight, based on the total weight of the medicament or a pharmaceutical composition. In still yet other embodiments, the mAb is present at a level of at least 25% by weight, based on the total weight of the medicament or a pharmaceutical composition.

The pharmaceutically acceptable carrier may be any pharmaceutically acceptable material or vehicle, such as a liquid or solid filler, diluent, excipient, solvent or encapsulating material, that is useful in carrying or transporting the active agents (e.g., the present mAb) from one organ, or portion of the body, to another organ, or portion of the body. The carrier must be “acceptable” in the sense of being compatible with the other ingredients of the formulation and is selected to minimize any degradation of the active agent and to minimize any adverse side effects in the subject. Depending on desired purposes, the medicament or a pharmaceutical composition of the present disclosure may further comprise one or more pharmaceutically-acceptable additives, including binder, flavoring, buffering agent, thickening agent, coloring agent, anti-oxidant, diluent, stabilizer, buffer, emulsifier, dispersing agent, suspending agent, antiseptic and the like.

The choice of a pharmaceutically acceptable carrier to be used in conjunction with the present mAb is basically determined by the way the medicament or a pharmaceutical composition being administered. The medicament or a pharmaceutical composition of the present invention may be administered to a subject via subcutaneous, intravenous, or intraarterial injection.

The medicament or a pharmaceutical composition for administration by injection may be prepared in a sterile aqueous or non-aqueous solution, suspension, and emulsion. Examples of the non-aqueous solution include, but are not limited to, propylene glycol, polyethylene glycol, vegetable oils such as olive oil, and injectable organic esters such as ethyl oleate. Illustrative examples of the aqueous solution include water, alcoholic solution, emulsion, or suspension, such as saline and buffered media. Common parenteral vehicles include sodium chloride solution, Ringer's dextrose, dextrose, sodium chloride, lactated Ringer's, or fixed oils; whereas intravenous vehicles often include fluid and nutrient replenishes, electrolyte replenishes (such as those based on Ringer's dextrose), and the like.

### (v) Uses of the Present Antibody, Pharmaceutical Composition, and Medicament

Also disclosed herein is a method of treating cachexia in a subject. The method comprises administering to the subject an effective amount of the mAb, pharmaceutical composition, or medicament in accordance with any aspect, embodiment of example of the present disclosure.

Depending on the desired purpose, the cachexia may be caused by and/or associated with cancer, CKD, kidney failure, heart failure, infectious disease (e.g., bacterial, viral, fungal, or parasitic infection), multiple sclerosis, tuberculosis, rheumatoid arthritis, cystic fibrosis, or COPD. In one exemplary embodiment, the cachexia is caused by and/or associated with a cancer (i.e., cancer cachexia (CC), which is caused by systemic inflammation and is characterized as a chronic inflammatory syndrome). Preferably, the cancer cachexia is caused by and/or associated with a PTHrP-expressing cancer (i.e., the cancer expressing or secreting PTHrP); for example, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

In one exemplary embodiment, the cachexia is caused by and/or associated with CKD or kidney failure. According to this embodiment, the administration of the present mAb, pharmaceutical composition, or medicament exhibits a therapeutic effect on CDK-/kidney failure-related cachexia.

In another exemplary embodiment, the cachexia is caused by and/or associated with heart failure. According to this embodiment, the administration of the present mAb, pharmaceutical composition, or medicament exhibits a therapeutic effect on cardiac dysfunction-/heart failure-related cachexia.

In still another exemplary embodiment, the cachexia is caused by and/or associated with tuberculosis. According to the embodiment, the administration of the present mAb, pharmaceutical composition, or medicament exhibits a therapeutic effect on tuberculosis-related cachexia.

According to some embodiments, the subject is a mouse, in which 0.1 to 100 mg/Kg of the present mAb is administered to the subject, such as 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/kg. Preferably, about 1 mg/kg to 50 mg/kg of the present mAb is administered to the subject. More preferably, about 5 mg/kg to 20 mg/kg of the present mAb is administered to the subject. According to one working example, about 10 mg/kg of the present mAb is sufficient to elicit a therapeutic effect in the subject (i.e., increasing the body weight of the subject).

A skilled artisan may readily determine the human equivalent dose (HED) of the present mAb, based on the doses determined from animal studies provided in working examples of this application. Accordingly, the effective amount of the present mAb suitable for use in a human subject may be in the range of 10 μg/kg to 10 mg/kg body weight for human; such as 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 μg/kg, or 1, 2, 3, 4, 5, 6, 7, 8, 9 or 10 mg/kg. Preferably, the HED is about 100 μg/kg to 5 mg/kg body weight. The dose can be administered in a single aliquot, or alternatively in more than one aliquot. The skilled artisan or clinical practitioner may adjust the dosage or regime in accordance with the physical condition of the patient or the severity of the diseases.

Depending on desired purpose, the present mAb, pharmaceutical composition or medicament may be administered to the subject 1 or 2 times every week, once every 2 weeks, once every 3 weeks, once every 4 weeks, once every 5 weeks, once every 6 weeks, once every 7 weeks, once every 8 weeks, or longer. The progress of this therapy is easily monitored by conventional techniques and assays. As could be appreciated, the dosing regimen can vary over time. According to an exemplary embodiment, the present mAb is administered to the subject once every week.

The present mAb, pharmaceutical composition or medicament may be administered to the subject by an appropriate route, such as intravenous, intraarterial, or intraperitoneal injection. According to some preferred embodiments of the present disclosure, the present mAb, pharmaceutical composition or medicament is administered to the subject via intravenous injection.

As would be appreciated, the present method can be applied to the subject, alone or in combination with additional therapies that have some beneficial effects on the prevention or treatment of cancer cachexia. Depending on the intended/therapeutic purpose, the present method can be applied to the subject before, during, or after the administration of the additional therapies.

According to certain embodiments of the present disclosure, the administration of the present mAb, pharmaceutical composition or medicament increases the body weight of the subject. According to some embodiments of the present disclosure, the administration of the present mAb, pharmaceutical composition or medicament decreases the loss of skeletal muscle and/or adipose tissues of the subject. According to various embodiments of the present disclosure, the administration of the present mAb, pharmaceutical composition or medicament increases the expression levels of albumin and pre-albumin (the serum markers reflecting the nutritional status of the subject), insulin-like growth factor-1 (IGF-1; a factor enhancing the anabolic process in skeletal muscle), and testosterone (a hormone known to suppress muscle proteolysis, reducing systemic inflammatory cytokines, stimulating anti-inflammatory cytokines, and stimulating protein anabolism in muscles); and decreases the expression levels of C-reactive protein (CRP; an inflammatory marker associated with cachexia), inflammatory cytokines (including tumor necrosis factor-alpha (TNF-α) and interleukine-6 (IL-6)), and cortisol (a hormone marker associated with protein breakdown) of the subject. According to some embodiments of the present disclosure, the administration of the present mAb, pharmaceutical composition or medicament decreases the resting energy expenditure (REE), and glucose metabolism of the subject.

Basically, the subject treatable by the present method is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Cell Culture

Rat osteosarcoma cell line UMR-106 cells (Bioresource Collection and Research Center (BCRC) number: 60270) were cultured in Dulbecco's Modified Eagle Medium (DMEM) supplemented with 10% fetal bovine serum (FBS). Human non-small cell lung cancer cell line HARA-B cells (Japanese Collection of Research Bioresources Cell Bank (JCRB) number: 1080.1) and human kidney cancer cell line 786-O cells (BCRC number: 60243) were grown in RPMI medium supplemented with 10% FBS. Human pancreatic cancer BxPC3 cells (JCRB number: 1448) were cultured in RPMI medium supplemented with 10% FBS.

Preparation of Murine mAbs 16G2 and 5B3

The murine monoclonal antibodies were produced via mice immunization. Specifically, the BALB/c mice were immunized with an Escherichia coli (E. coli)-expressed fusion protein comprising a PTHrP peptide and a polyhistidine-tag (His-tag) fused to the C-terminus of the PTHrP peptide. Post-immunization, B cells were extracted from the spleens of the immunized mice, followed by fusing with myeloma cells. The titers of the antibodies secreted by the thus-produced hybridomas were determined by ELISA so as to identify the hybridoma clones (clones 16G2 and 5B3) that expressed the antibodies exhibiting binding affinity and specificity towards PTHrP. The selected clones 16G2 and 5B3 were then cultured to facilitate antibody production. The antibodies isolated from the supernatant of the culture medium of clones 16G2 and 5B3 were purified and then subjected to sequence analysis and activity assays.

The thus-produced antibodies were respectively designated as “murine mAb 16G2” and “murine mAb5B3”, in which the VH and VL sequences of the murine antibodies were summarized in Table 1.

TABLE 1
VH and VL sequences of murine mAbs
16G2 and 5B3

| | | SEQ ID |
|---|---|---|
| Name | Amino acid sequence | NO |
| mAb 16G2 VH | QVQLQQPGAELGMPGASVKMSCKAS | 7 |
| | GYIFTDYWMHWVKQRPGQGLEWIGA | |
| | IDTSDSYSSYNQKFQGKATLTVDES | |
| | SSTAYMQLSSLTSEDSAVYYCTLGD | |
| | YWGQGTTLTVSS | |
| mAb 16G2 VL | DVVMTQTPLTLSVTIGQPASMSCKS | 8 |
| | SQSLLESDGKTYLNWLLQRPGQSPK | |
| | RLIYLVSKLDSGVPDRFTGSGSGTD | |
| | FTLKISRVEAEDLGVYYCCQGTHFP | |
| | WTFGGGTKLEIK | |
| mAb 5B3 VH | QVQLQQPGAELVMPGASVKMSCKAS | 16 |
| | GYTFTDYWMHWVKQRPGQGLEWIGA | |
| | LDTSDSYASYNQRFKGKATLTVDAS | |
| | SSTAYMQLSSLTSEDSAVYYCTLGD | |
| | YWGQGTTLTVSS | |
| mAb 5B3 VL | DVVMTQTPLTLSVTFGQPASISCKS | 17 |
| | SQSLLESDGKTYLNWLLQRPGQSPK | |
| | RLIYLVSKLDSGVPDRFTGSGSGTD | |
| | FTLKISRVEAEDLGVYYCCQGTHFP | |
| | WTFGGGTKLEIK | |

* The CDR sequences (including CDR-H1, CDR-H2 and CDR-H3 of VH domain, and CDR-L1, CDR-L2 and CDR-L3 of VL domain) were marked in bold, in sequence.

Preparation of Chimeric mAbs 16G2 and 5B3

The nucleotide sequence encoding the VH and VL domains of murine mAbs 16G2 and 5B3 were, respectively, cloned into the expression vector pcDNA3.4, which contained a constant domain of human IgG. The vector containing both non-human variable and human constant domains was transfected into host cells EXPICHO-S™ for expression. The chimeric monoclonal antibodies were isolated from the supernatant of the culture medium, followed by purification and sequence analysis.

The thus-produced chimeric mAb comprised the VH and VL domains of murine mAb (i.e., the VH and VL domains of murine mAb 16G2 or murine mAb 5B3), and the constant domains of a human antibody, in which the CL domain comprised the amino acid sequence of SEQ ID NO: 18, and the CH domain comprised the amino acid sequence of SEQ ID NO: 19.

### Preparation of Humanized mAb 16G2

The mouse-derived framework regions within the VH and VL domains of the chimeric mAb were first identified and replaced by human framework regions while preserving critical binding areas. Structural and functional assessments were employed to ensure the successful humanization of these regions. Subsequently, the modified variable domains were expressed in host cells EXPICHO-S™ and optimized for maximum production of the humanized mAb. After purification, the humanized mAbs were subjected to sequence analysis and activity assays.

Four humanized VH domains (including VH1, VH2, VH3 and VH4 domains) and three humanized VL domains (including VL1, VL2 and VL3 domains) were produced in this study. The amino acid sequences of the humanized VH and VL domains were summarized in Table 2.

TABLE 2
Amino acid sequences of humanized VH and
VL domains

| | | SEQ ID |
|---|---|---|
| Name | Amino acid sequence | NO |
| VH1 | QVQLVQSGAEVKKPGASVKVSCKAS | 9 |
| | GYIFTDYWMHWVRQAPGQGLEWMGA | |
| | IDTSDSYSSYNQKFQGRVTMTRDTS | |
| | TSTVYMELSSLRSEDTAVYYCTLGD | |
| | YWGQGTTVTVSS | |
| VH2 | QVQLVQSGAEVKKPGASVKVSCKAS | 10 |
| | GYIFTDYWMHWVRQAPGQGLEWIGA | |
| | IDTSDSYSSYNQKFQGRATMTVDTS | |
| | TSTVYMELSSLRSEDTAVYYCTLGD | |
| | YWGQGTTVTVSS | |
| VH3 | QVQLVQSGAEVKKPGASVKVSCKAS | 11 |
| | GYIFTDYWMHWVKQAPGQGLEWIGA | |
| | IDTSDSYSSYNQKFQGRATMTVDTS | |
| | TSTVYMELSSLRSEDTAVYYCTLGD | |
| | YWGQGTTVTVSS | |
| VH4 | QVQLVQSGAEVKKPGASVKVSCKAS | 12 |
| | GYIFTDYWMHWVKQAPGQGLEWIGA | |
| | IDTSDSYSSYNQKFQGRATMTVDES | |
| | TSTVYMELSSLRSEDTAVYYCTLGD | |
| | YWGQGTTVTVSS | |
| VL1 | DVVMTQSPLSLPVTLGQPASISCKS | 13 |
| | SQSLLESDGKTYLNWFQQRPGQSPR | |
| | RLIYLVSKLDSGVPDRFSGSGSGTD | |
| | FTLKISRVEAEDVGVYYCCQGTHFP | |
| | WTFGGGTKLEIK | |
| VL2 | DVVMTQSPLSLPVTLGQPASISCKS | 14 |
| | SQSLLESDGKTYLNWLLQRPGQSPR | |
| | RLIYLVSKLDSGVPDRFSGSGSGTD | |
| | FTLKISRVEAEDVGVYYCCQGTHFP | |
| | WTFGGGTKLEIK | |
| VL3 | DVVMTQSPLSLPVTLGQPASMSCKS | 15 |
| | SQSLLESDGKTYLNWLLQRPGQSPK | |
| | RLIYLVSKLDSGVPDRFSGSGSGTD | |
| | FTLKISRVEAEDVGVYYCCQGTHFP | |
| | WTFGGGTKLEIK | |

* The CDR sequences (including CDR-H1, CDR-H2 and CDR-H3 of VH domain, and CDR-L1, CDR-L2 and CDR-L3 of VL domain) were marked in bold, in sequence.

The humanized mAbs containing the humanized VH and VL domains were respectively designated as “humanized mAb 16G2-1” (the mAb comprising humanized VH1 and VL1 sequences), “humanized mAb 16G2-2” (the mAb comprising humanized VH1 and VL2 sequences), “humanized mAb 16G2-3” (the mAb comprising humanized VH1 and VL3 sequences), “humanized mAb 16G2-4” (the mAb comprising humanized VH2 and VL1 sequences), “humanized mAb 16G2-5” (the mAb comprising humanized VH2 and VL2 sequences), “humanized mAb 16G2-6” (the mAb comprising humanized VH2 and VL3 sequences), “humanized mAb 16G2-7” (the mAb comprising humanized VH3 and VL1 sequences; also named as “BGM-2121” in the study), “humanized mAb 16G2-8” (the mAb comprising humanized VH3 and VL2 sequences), “humanized mAb 16G2-9” (the mAb comprising humanized VH3 and VL3 sequences), “humanized mAb 16G2-10” (the mAb comprising humanized VH4 and VL1 sequences), “humanized mAb 16G2-11” (the mAb comprising humanized VH4 and VL2 sequences), and “humanized mAb 16G2-12” (the mAb comprising humanized VH4 and VL3 sequences).

### Surface Plasmon Resonance (SPR)

The affinity of antigen to antibodies were measured using BIACORE™ SPR Systems. In brief, antibodies were injected on the sensor chip and immobilized with mouse antibody capture kit as capture. Antigen was diluted into multiple concentrations (50, 25, 12.5, 6.25, 3.125, 1.5625 nM) and injected over the surface of flow cell 1 and 2 as association phase, followed by injecting HBS-EP+ running buffer (HEPES 10 mM, NaCl 150 mM, EDTA 3 mM, 0.005% TWEEN® 20) as dissociation phase. All the data were recorded at 25° C. and processed using software.

### Enzyme Linked Immunosorbent Assay (ELISA)

The biotinylated PTHrP fragment containing the 34 N-terminal residues of human PTHrP (hereinafter as “PTHrP (1-34) peptide”) and biotinylated parathyroid hormone (PTH) fragment containing the 34 N-terminal residues of human PTH (hereinafter as “PTH (1-34) peptide”) were respectively diluted to a final concentration of 0.2 μg/mL, and then added to a 96-well plate. The plate was incubated at room temperature for 2 hours. After the washing and blocking steps, the diluted antibody was added to the wells, followed by incubating at room temperature for 2 hours. Next, a secondary antibody was added to the wells. After further washes, TMB (3,3′,5,5′-Tetramethylbenzidine) substrate was added to induce color development. The reaction was halted using HCL, and the absorbance was measured at 450 nm with a reference at 570 nm. The resulting absorbance data was analyzed by software via nonlinear regression for determining the half maximal effective concentration (EC50).

### Cell Based-Neutralizing Assay

For cell-base CAMP neutralization assay, UMR-106 cells were seeded into a 96-well plate at a density of 1.5×10⁴ cells per well and incubated at 37° C. for 3 days. Then, the medium was removed and washed by serum-free medium and then cells were incubated with experimental medium (DMEM containing 10 mM HEPES and 0.5 mM isobutylmethylxanthine (IBMX)) at 37° C. for 30 minutes. The experimental medium was removed, and recombinant PTHrP (1-34) peptide (20 ng/mL) with or without anti-PTHrP antibody (4× series dilution: from 2.5 μg/mL to 0.04 μg/mL) were added and then incubated at 37° C. for 30 minutes. After incubation, the medium was aspirated and treated with 250 μl 0.1 M HCL at room temperature for 20 minutes. Cells were harvested and transferred to a V-bottom 96-well dish followed by centrifuging at 1,000×g for 10 minutes at 4° C. The supernatant was transferred into a clean test tube. The absorbance was measured at a wavelength between 405 and 420 nm using an ELISA reader.

### In Vitro Neutralizing Assay (Competition ELISA)

PTHIR protein (1 μg/mL) in coating buffer was added into ELISA plate and incubated overnight at 4° C., followed by washing with PBS-T washing buffer (1× PBS containing 0.05% TWEEN® 20). The protein coating was blocked by incubation with blocking buffer (washing buffer containing 2% (w/v) bovine serum albumin (BSA)) at 37° C. for 1.5 hours. Biotinylated PTHrP was diluted to 0.5 μg/mL with dilution buffer (washing buffer containing 0.5% (w/v) BSA) and anti-PTHrP antibody was diluted to 40, 10, 2.5 and 0.625 μg/mL with dilution buffer. The mixture of biotinylated PTHrP and anti-PTHrP antibodies were added into the plate and incubated at 37° C. for 1 hour. Next, 100 μL of streptavidin-HRP (0.1 μg/mL) working solution was added into the plate and incubated at 37° C. for 1 hour, and then added 100 μL TMB substrate working solution to each well. After 10 minutes, 100 μL of 1N HCl were added to stop the reaction. The absorbance was measured at a wavelength 450 nm using an ELISA reader.

### Animal Models

### (i) Intrasplenic Xenograft Model

Twenty-one male NPG mice were randomly divided into three groups: the control (G1), BxPC3 cells with hIgG Ab (G2), and BxPC3 cells with 10 mg/kg present mAb (G3). Each mouse in G2 and G3 groups received a splenic injection of 2.5×10⁶ BxPC3 cells on the first experimental day (Day 0). In addition, mice in G1 group were injected with the serum-free RPMI-1640 medium. Human IgG Ab (hIgG Ab) for the G2 group and present mAb for the G3 group were administered intravenously once weekly for four weeks after 7 days of tumor cell inoculation. Body weights were measured twice weekly until Day 40, and submandibular blood samples from all three groups at multiple time points to obtain serum samples. At the endpoint, animals were euthanized to observe the liver metastasis and tumor growth in pancreas.

### (ii) NSCLC-Induced Cachexia Model

5×10⁶ HARA-B cells were suspended in 75 μL RPMI1640 (no phenol red) with 75 μL Matrigel® and subcutaneously injected into the dorsal flanks of four to six-week-old nude mice. After inoculation, the mice were randomly divided into three groups respectively receiving medium only (Healthy control), anti-PTHrP antibody (10 mg/kg, once per week) and control hIgG (10 mg/kg, once per week) via intravenous (IV.) injection (Table 3) for four consecutive weeks. Prior to first dosing and before euthanasia, whole-body microcomputed tomography (hereinafter “microCT”) imaging was performed to monitor changes in muscle and fat composition. Tumor volume was determined by measuring tumor length (L) and width (W) using calipers and calculated by the formula (length×width×width/2). The serum was collected for calcium measurements once per week. Mice were sacrificed by CO₂ euthanasia at the end of the study, and the connective tissue around the tumor was resected. The tumor tissues were weighed and photographed, then stored in 10% formaldehyde at room temperature. The skeletal muscles, including the gastrocnemius (GA), soleus (SOL), tibialis anterior (TA), and quadriceps femoris (QF), of the mice were also collected at the experimental endpoint of the study. The harvested tissues were fixed in 4% paraformaldehyde, processed for paraffin embedding, sectioned at a thickness of 4-6 μm, hematoxylin and eosin (H&E) stained, and photographed for histological morphology analysis. The images of the cross-sectional area of muscle fibers were quantified using ImageJ software (NIH, Bethesda, MD, USA). Results were presented as the mean and standard error of the mean (Mean±SEM). Comparisons of all data collected for each treatment group with concurrent negative control data will be calculated using a t-test. p≤0.05 is considered significant.

TABLE 3
Experimental design of NSCLC-induced cachexia model

| | | | Dose of | | |
|---|---|---|---|---|---|
| | Test | Route of | injection | Fre- | Animal |
| Groups | materials | administration | (mg/kg) | quency | number |
| G1: Healthy | Medium | IV. | - | Once/ | 5 |
| control | | | | week | |
| G2: hIgG | hIgG | IV. | 10 | Once/ | 8 |
| | | | | week | |
| G3: BGM- | BGM-2121 | IV. | 10 | Once/ | 8 |
| 2121 | | | | week | |

(iii) Kidney Cancer-Induced Cachexia Model

6×10⁶ 786-O cells were suspended in 100 μL RPMI1640 (no phenol red) with 100 μL Matrigel® and subcutaneously injected into the dorsal flanks of four to six-week-old nude mice. After inoculation, the mice were randomly divided into three groups respectively receiving medium only (Healthy control), anti-PTHrP antibody (10 mg/kg, once per week) and control hIgG (10 mg/kg, once per week) via intravenous (IV.) injection (Table 4) for four consecutive weeks. Tumor volume was determined by measuring tumor length (L) and width (W) using calipers and calculated by the formula (length×width×width/2). The serum was collected for calcium measurements once per week. Mice was sacrificed by CO₂ euthanasia at the end of the study, and the connective tissue around the tumor was resected. The tumor tissues were weighed and photographed, then stored in 10% formaldehyde at room temperature. Results were presented as the mean and standard error of the mean (Mean±SEM). Comparisons of all data collected for each treatment group with concurrent negative control data will be calculated using a t-test. p≤0.05 is considered significant. The skeletal muscles, including the gastrocnemius (GA), soleus (SOL), tibialis anterior (TA), and quadriceps femoris (QF), and the adipose tissues, including inguinal white adipose tissue (iWAT), epididymal white adipose tissue (eWAT), and (iBAT), of the mice were also collected at the experimental endpoint of the study. The harvested tissues were fixed in 4% paraformaldehyde, processed for paraffin embedding, sectioned at a thickness of 4-6 μm, hematoxylin and eosin (H&E) stained, and photographed for histological morphology analysis. The images of the cross-sectional area of muscle fibers and adipocytes were quantified using ImageJ software (NIH, Bethesda, MD, USA).

TABLE 4
Experimental design of kidney cancer-induced cachexia model

| | | | Dose of | | |
|---|---|---|---|---|---|
| | Test | Route of | injection | Fre- | Animal |
| Groups | materials | administration | (mg/kg) | quency | number |
| G1: Healthy | Medium | IV. | - | Once/ | 5 |
| control | | | | week | |
| G2: hIgG | hIgG | IV. | 10 | Once/ | 8 |
| | | | | week | |
| G3: BGM- | BGM-2121 | IV. | 10 | Once/ | 8 |
| 2121 | | | | week | |

### Example 1: Characterization of the Present mAb

### 1.1 Binding Affinity

The binding activities of the present mAb to PTHrP were evaluated in this example. As described in “Materials and Methods” of the present disclosure, the present mAb (i.e., the murine mAb, chimeric mAb or humanized mAb) and PTHrP antigen were respectively added to sensor chips, and the binding activity therebetween was determined by SPR. The results were summarized in Tables 5 and 6.

The data of Table 5 indicated that all tested mAb (including murine mAb 16G2, murine mAb 5B3, chimeric mAb 16G2 and chimeric mAb 5B3) recognized and bound to PTHrP, in which compared to the murine mAbs and reference Ab (i.e., Chugai antibody, serving as a positive control), the chimeric antibodies exhibited higher binding affinity to PTHrP (the KD values of chimeric 16G2 mAb and chimeric 5B3 mAb were 1.15×10⁻¹⁰ and 2.08×10⁻¹⁰, respectively).

TABLE 5
Antigen-binding affinity of specified mAbs

| | Ligand | Analyte | ka (1/Ms) | kd (1/s) | KD (M) | Rmax (RU) | Chi² (RU²) |
|---|---|---|---|---|---|---|---|
| Mouse | 16G2 | peptide | 2.14E+06 | 1.97E−03 | 9.17E−10 | 19.43 | 0.00866 |
| | 5B3 | peptide | 6.61E+05 | 4.38E−04 | 6.63E−10 | 13.04 | 0.00692 |
| | Positive control | peptide | 1.14E+06 | 6.05E−04 | 5.29E−10 | 10.84 | 0.00975 |
| | (reference Ab) | | | | | | |
| Chimeric | 16G2 | peptide | 2.64E+06 | 3.04E−04 | 1.15E−10 | 32.73 | 0.0155 |
| | 5B3 | peptide | 1.48E+06 | 3.07E−04 | 2.08E−10 | 38.36 | 0.0159 |
| | Positive control | peptide | 2.88E+06 | 1.80E−03 | 6.25E−10 | 54.56 | 0.0481 |
| | (reference Ab) | | | | | | |

Ka: The association rate constant.
Kd: The dissociation rate constant.
KD: The equilibrium dissociation constant; KD = kd/ka.

The data of Table 6 demonstrated the binding affinity of humanized mAbs (including humanized mAb 16G2-1 to humanized mAb 16G2-12) to PTHrP.

TABLE 6
Antigen-binding affinity of specified humanized mAbs

| Ligand | Analyte | Chi² (RU²) | ka (1/Ms) | kd (1/s) | KD (M) | Rmax (RU) |
|---|---|---|---|---|---|---|
| Reference Ab | naïve peptide | 1.70E−02 | 1.97E+06 | 6.03E−03 | 3.06E−09 | 46 |
| Murine 16G2 | naïve peptide | 3.63E−02 | 2.99E+06 | 2.87E−04 | 9.60E−11 | 31.2 |
| mAb 16G2-1 | naïve peptide | 2.80E−02 | 8.61E+05 | 1.27E−04 | 1.48E−10 | 42.2 |
| mAb 16G2-2 | naïve peptide | 1.79E−02 | 1.28E+06 | 5.27E−04 | 4.13E−10 | 41 |
| mAb 16G2-3 | naïve peptide | 4.07E−01 | 1.58E+06 | 1.72E−03 | 1.09E−09 | 42.1 |
| mAb 16G2-4 | naïve peptide | 4.35E−02 | 1.08E+06 | 1.51E−04 | 1.39E−10 | 37.2 |
| mAb 16G2-5 | naïve peptide | 1.04E−02 | 1.19E+06 | 6.04E−04 | 5.09E−10 | 44.7 |
| mAb 16G2-6 | naïve peptide | 1.57E−02 | 1.55E+06 | 6.27E−04 | 4.04E−10 | 38 |
| mAb 16G2-7 | naïve peptide | 1.47E−02 | 1.18E+06 | 1.90E−04 | 1.60E−10 | 36 |
| (BGM-2121) | | | | | | |
| mAb 16G2-8 | naïve peptide | 1.97E−02 | 1.46E+06 | 2.50E−04 | 1.72E−10 | 41.5 |
| mAb 16G2-9 | naïve peptide | 4.51E−02 | 2.37E+06 | 4.35E−04 | 1.84E−10 | 34.5 |
| mAb 16G2-10 | naïve peptide | 1.10E−02 | 1.16E+06 | 1.02E−04 | 8.83E−11 | 49.5 |
| mAb 16G2-11 | naïve peptide | 1.36E−02 | 1.42E+06 | 2.94E−04 | 2.08E−10 | 60.4 |
| mAb 16G2-12 | naïve peptide | 1.36E−02 | 1.84E+06 | 1.68E−04 | 9.11E−11 | 38.1 |

Ka: The association rate constant.
Kd: The dissociation rate constant.
KD: The equilibrium dissociation constant; KD = kd/ka.
Reference Ab: Chugai antibody; serving as a positive control.
Murine 16G2: Murine mAb 16G2 comprising the VH domain of SEQ ID NO: 7 and the VL domain of SEQ ID NO: 8.

The binding affinities of the chimeric mAb 16G2 and mAb 16G2-7 (BGM-2121) toward PTHrP (1-34) peptide or PTH (1-34) peptide were further confirmed by ELISA. According to the result of Table 7, the chimeric mAb 16G2 and BGM-2121 recognized and bound to the PTHrP (1-34) peptide with equilibrium dissociation constant (KD) of 2.44 nM and 1.79 nM, respectively. It was noted that the chimeric mAb 16G2 as well as BGM-2121 exhibited no off-target binding to the PTH (1-34) fragment, the primary regulator of calcium and phosphate metabolism in bone and kidney.

TABLE 7
Binding affinity (EC50) of specified antibodies
towards PTHrP (1-34) or PTH (1-34) peptide

| Peptide | Chimeric mAb 16G2 | | BGM-2121 | | anti-hPTH** | |
|---|---|---|---|---|---|---|
| PTH (1-34) | >1 | mM | >1 | mM | 3.06 | nM |
| PTHrP (1-34) | 2.44 | nM | 1.79 | nM | >1 | mM |

**Anti-hPTH: a commercial antibody developed by Phoenix Pharmaceuticals that exhibited a high binding affinity toward human PTH (1-34) peptide; serving as a control group in the study.

### 1.2 In Vitro Activity

In this example, the bio-activity of the present mAb was analyzed by neutralization assay. The results were summarized in Tables 8 and 9.

Cell-based neutralization assay was performed using the rat osteosarcoma cell line UMR-106, which expresses endogenous PTH1 receptor. All tested mAb (including murine mAb 16G2, murine mAb 5B3, chimeric mAb 16G2 and chimeric mAb 5B3) inhibited the PTHrP-induced cAMP production in UMR-106 cells (data not shown). According to the results of Table 8, both chimeric mAb 16G2 (with an EC₅₀ of 0.81 nM) and BGM-2121 (with an EC₅₀ of 0.89 nM) exhibited high effectiveness in selectively inhibiting PTHrP (1-34) peptide-induced cAMP production. Importantly, both chimeric mAb 16G2 and BGM-2121 exhibited no neutralizing activity against PTH (1-34) peptide-induced cAMP production (with an EC₅₀>40 nM).

TABLE 8
Neutralizing activity (EC50) of specified antibodies
by cell-based neutralization assay

| Peptide | Chimeric mAb 16G2 | | BGM-2121 | |
|---|---|---|---|---|
| hPTH (1-34) | >40 | nM | >40 | nM |
| PTHrP (1-34) | 0.81 | nM | 0.89 | nM |

The data of Table 9 indicated that each of the present humanized mAbs 16G2-1, 16G2-4, 16G2-7 (BGM-2121) and 16G2-10 was useful in neutralizing the activity of PTHrP antigen, in which the humanized 16G2-10 mAb exhibited the highest competition neutralizing activity (IC50=1.36×10⁻⁸; Table 9), and the humanized 16G2-7 mAb exhibited the highest cell-base neutralizing activity (IC50=8.926×10⁻¹⁰; Table 9).

TABLE 9
The neutralizing the activity of specified humanized mAbs

| | | | | | Competition | Cell-base |
|---|---|---|---|---|---|---|
| | | | Back | | Neutralizing | Neutralizing |
| Item | Heavy chain | Light chain | mutation | KD (M) | activity (M) | activity (M) |
| Name | VH | VL | 0 | 1.15E−10 | 2.25E−08 | 8.147e−010 |
| mAb 16G2-1 | VH1 (h-2 back) | VL1 (h-0 back) | 2 | 1.29E−10 | 1.70E−08 | 1.206e−009 |
| mAb 16G2-4 | VH2 (h-5 back) | | 5 | 1.16E−10 | 1.95E−08 | 1.328e−009 |
| mAb 16G2-7 | VH3 (h-6 back) | | 6 | 8.79E−11 | 1.54E−08 | 8.926e−010 |
| mAb 16G2-1 | VH4 (h-7 back) | | 7 | 1.03E−10 | 1.36E−08 | 9.389e−010 |

| | | | | |
|---|---|---|---|---|
| R | Positive control | 3.06E−09 | 3.07E−08 | 7.852e−009 |
| | (reference Ab) | | | |

The data of Table 10 further confirmed that BGM-2121 was capable of binding to biotin-labeled PTHrP (1-34) peptide and competed with PTH1R binding. The result suggested that BGM-2121 exerted a neutralizing activity by interfering with the interaction between the biotin-labeled PTHrP (1-34) peptide and PTH1R receptor. According to the results of Table 8, both the chimeric mAb 16G2 antibody (with an EC₅₀ of 22.5 nM) and BGM-2121 (with an EC₅₀ of 15.4 nM) exhibited potent inhibition of human PTHrP (1-34) peptide.

TABLE 10
Neutralizing activity (EC50) of specified antibodies
by competition ELISA-based assay

| Name | Chimeric mAb 16G2 | BGM-2121 |
|---|---|---|
| PTHrP (1-34) | 22.5 nM | 15.4 nM |

These results demonstrated that each of the present mAb (including murine mAb, chimeric mAb and humanized mAb) exhibited PTHrP-binding affinity and may serve as a neutralizing antibody to treat cancers (especially PTHrP-positive cancers) via inhibiting the activity of PTHrP.

### Example 2: Effect of the Present mAb on Liver Metastasis and Tumor Growth in Pancreas

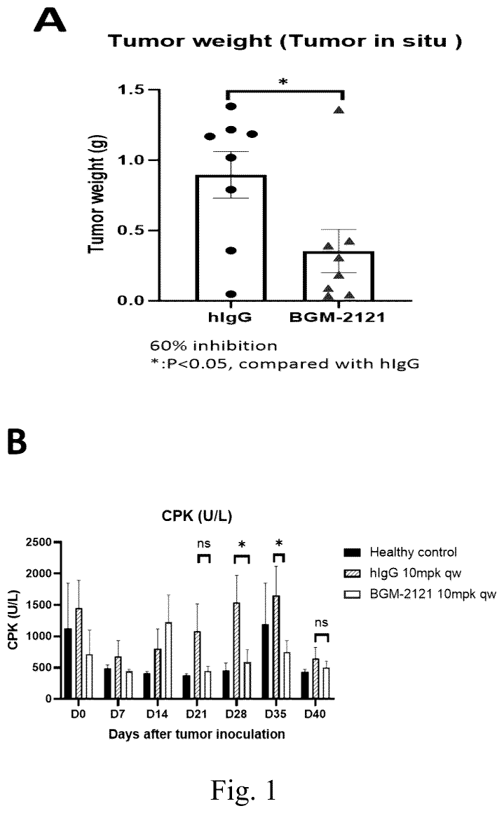

The effect of the present BGM-2121 on liver metastasis and tumor growth in pancreas was investigated in this example. As described in “Materials and Methods” of the present disclosure, pancreatic cancer cell line (i.e., BxPC-3) were injected into the spleen of mice, followed by the treatment with the present BGM-2121 or control antibody IgG. Results are illustrated in FIG. 1.

The growth of pancreatic cancer was successfully suppressed by the treatment of the BGM-2121, with a significant reduction in the tumor weight (about 60% reduction), as compared to that of a control group (i.e., hIgG-treated mice). Furthermore, treatment with BGM-2121 effectively mitigated muscle damage and liver metastasis, as reflected by significantly reduced creatine phosphokinase (CPK) levels relative to the hIgG control group at critical time points.

Taken together, data from this example demonstrated that the present BGM-2121 could effectively inhibit tumor growth while protecting the test subjects from muscle damage and liver metastasis.

### Example 3: Effect of the Present mAb on Cachexia in Animal Models

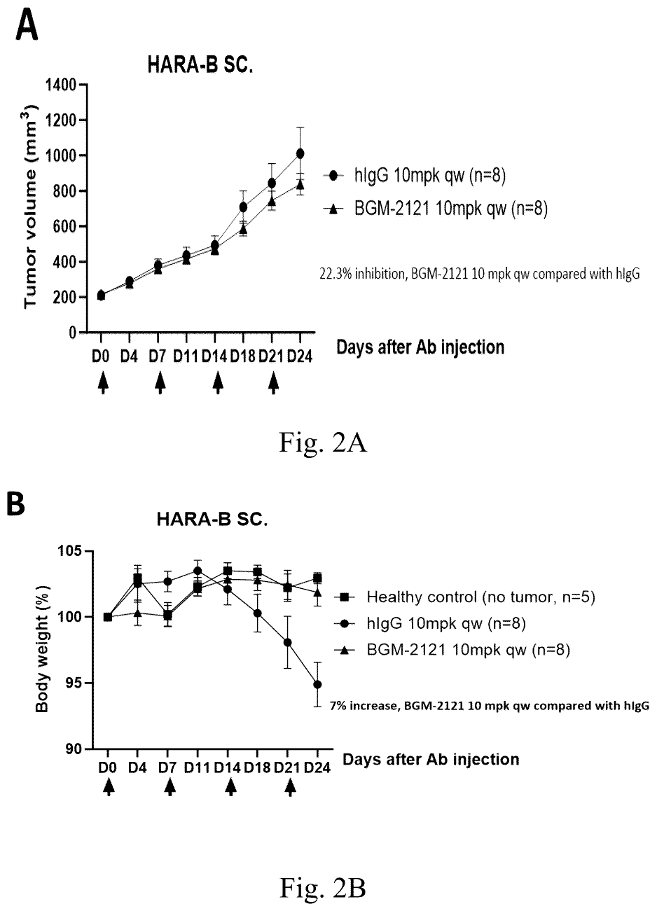

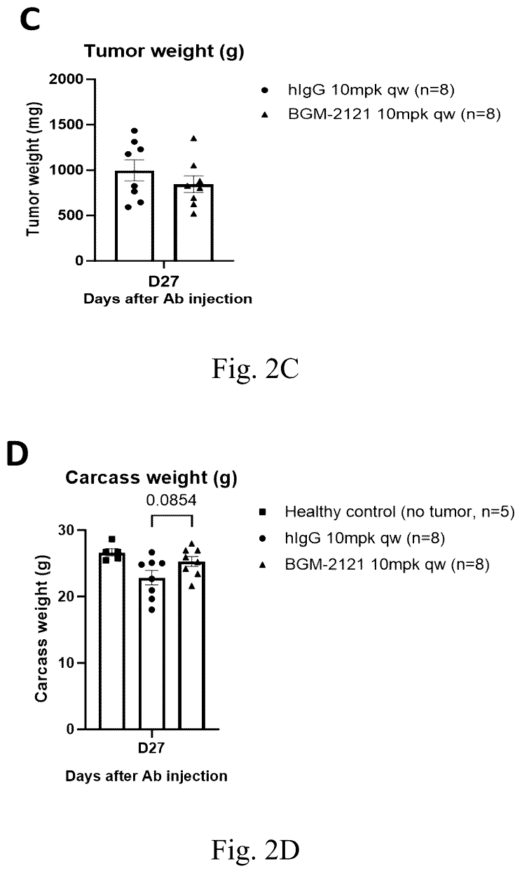

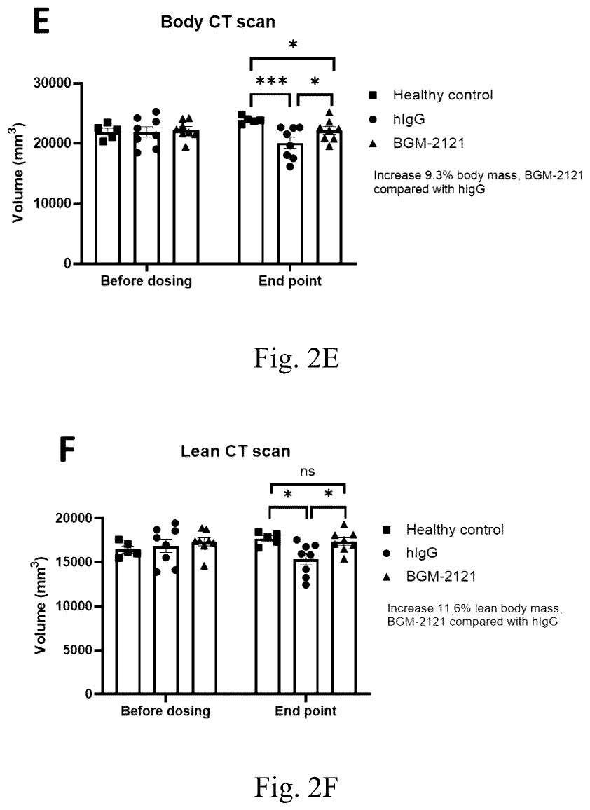

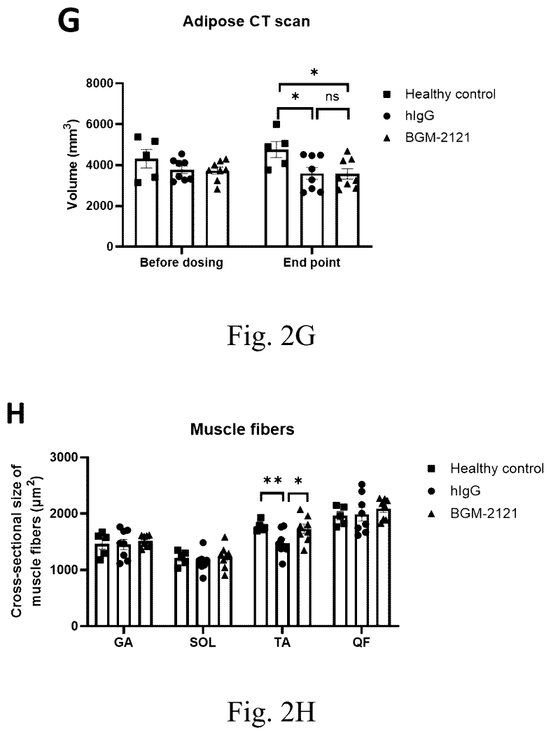

The effect of the present BGM-2121 on cancer cachexia was investigated in this example by measuring the changes in body weight and body compositions in lung cancer and kidney cancer animal models established according to procedures described in “Materials and Methods” of the present disclosure. Lung cancer cell line (i.e., HARA-B) and kidney cancer cell line (i.e., 786-O) were respectively injected into the dorsal flanks of mice, followed by the treatment with the present BGM-2121 or control antibody hIgG. The results are depicted in FIGS. 2 to 4.

### 3.1 Lung Cancer-Induced Cachexia

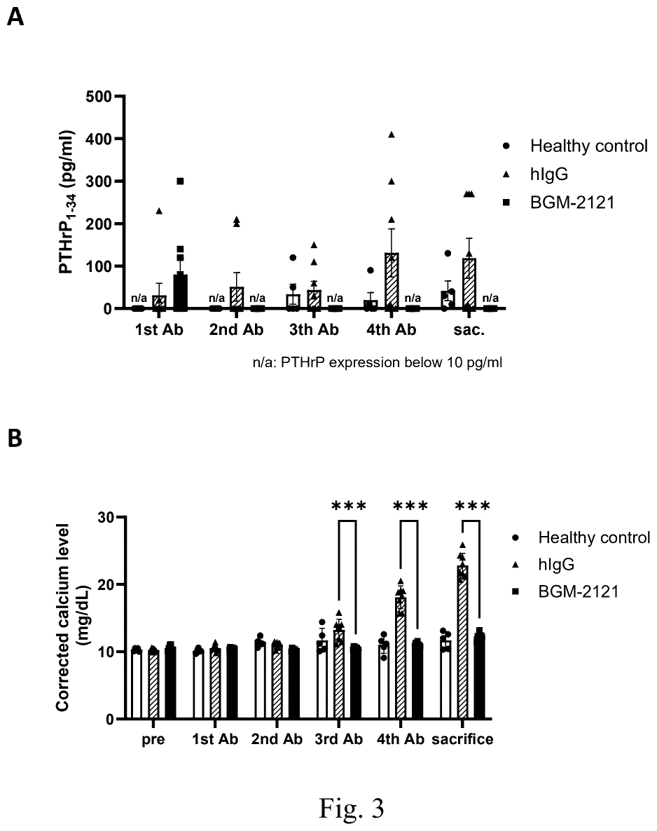

In general, cachexia in humans is regarded as having more than 5% body weight loss in 6 months or a body mass index (BMI) of less than 20. It was noted that HARA-B cells-bearing mice initiated body weight loss on day 18. By days 18-24, a marked reduction in body weight was observed, consistent with the development of cancer cachexia (FIG. 2B). The cancer cachexia was effectively reversed by the treatment with BGM-2121, with 22.3% inhibition in tumor volume and 15.4% in tumor weight (FIGS. 2A and 2C). Compared to the control hIgG group, tumor-bearing mice showed a 7% increase in body weight, indicating effective prevention of weight loss (FIGS. 2B and 2D, BGM-2121 group). MicroCT images further confirmed that BGM-2121 was more effective than hIgG in increasing whole body mass (9.3%) and lean body mass (11.6%) (FIGS. 2E and 2F); and was as effective as hIgG in keeping the adipose tissue mass (FIG. 2G) in cachexia mice. Histological analysis of skeletal muscle tissues revealed that, compared to the Health control group, both hIgG group and BGM-2121 group showed reductions in skeletal muscle fiber size, and BGM-2121, compared to Health control group and hIgG group, was more effective in mitigating and restoring muscular tissue loss (FIG. 2H). In addition, BGM-2121 also significantly decreased the secretion of hPTHrP (1-34) (FIG. 3A), thereby preventing the elevation of serum calcium levels (FIG. 3B).

### 3.2 Kidney Cancer-Induced Cachexia

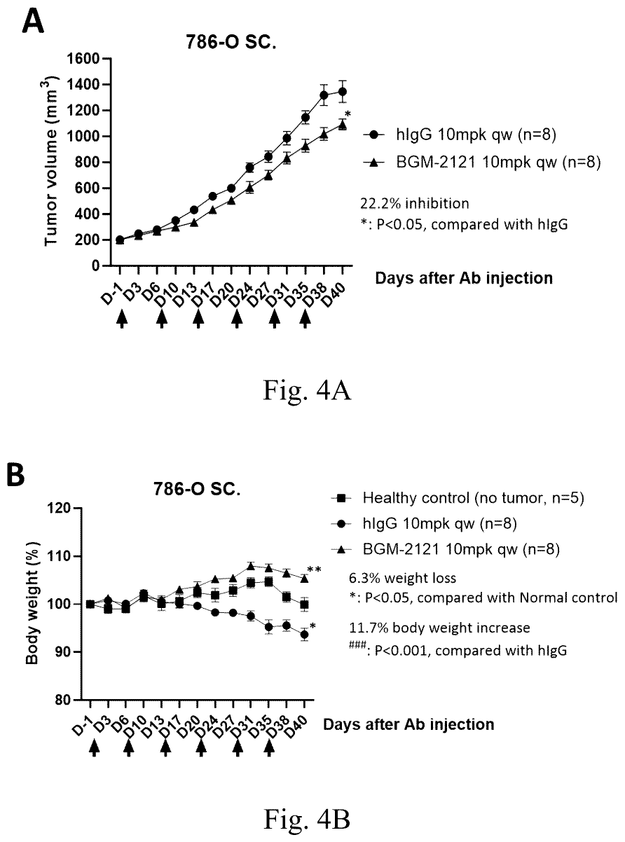

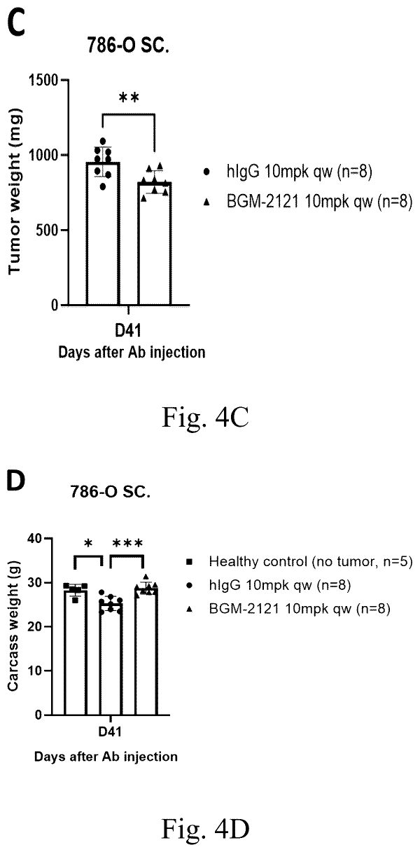

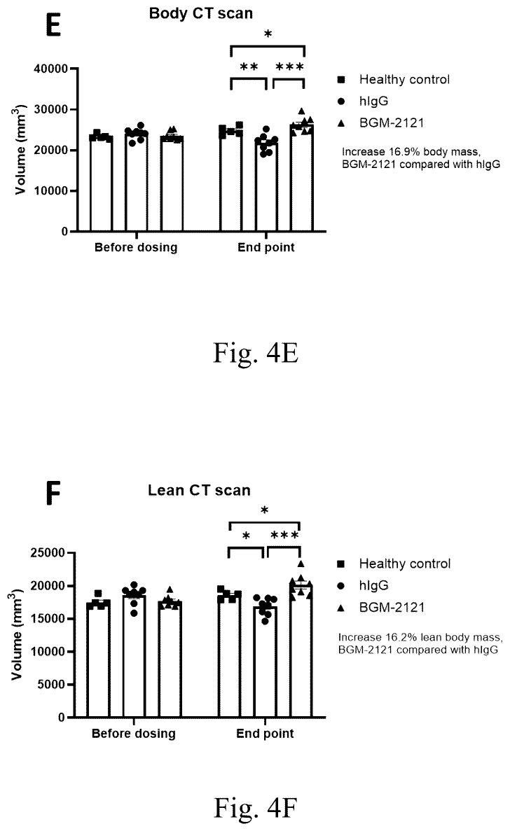

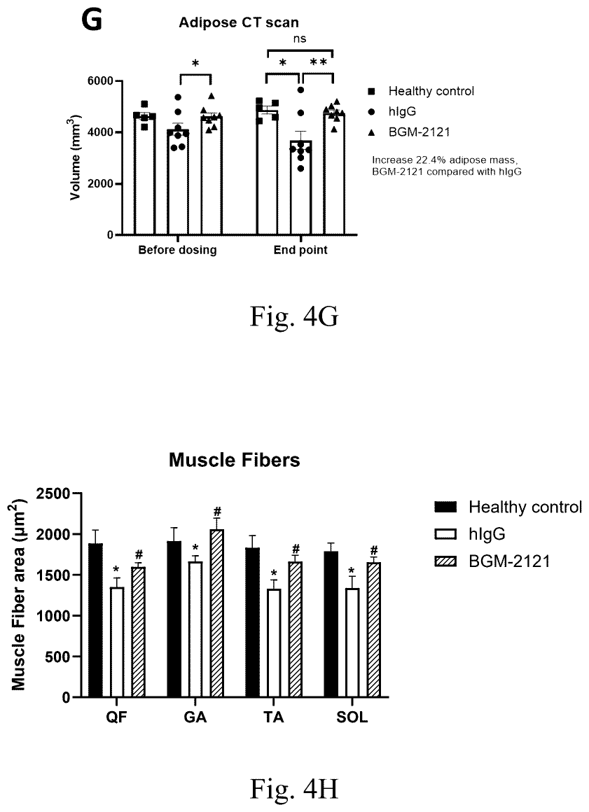

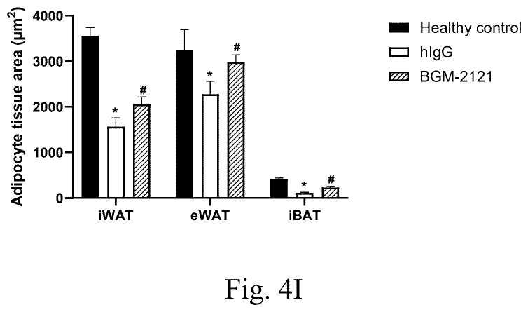

Similar results were also found in 786-O cells-bearing mice, which initiated body weight loss on day 20. By days 20-40, a marked reduction in body weight was observed, consistent with the development of cancer cachexia (FIG. 4B). The cancer cachexia was effectively reversed by the treatment of BGM-2121 with 22.2% inhibition in tumor volume and 30% inhibition in tumor weight (FIGS. 4A and 4C). Compared to the control hIgG group, tumor-bearing mice in BGM-2121 group showed a 11.7% increase in body weight, indicating effective prevention of weight loss (FIGS. 4B and 4D, BGM-2121 group). MicroCT images further confirmed that BGM-2121 was more effective in increasing whole body mass (16.9%), lean body mass (16.2%) and the adipose tissue mass (22.4%) as compared to that in hIgG treatment mice (FIGS. 4E to 4G). Histological analysis of skeletal muscles and adipose tissues also confirmed that, compared to hIgG, BGM-2121 was more effective in mitigating muscular and adipose tissues loss (FIGS. 4H and 4I).

Weight, skeletal muscle loss, and adipose tissue loss are often associated with advanced malignancies. Unfortunately, there are no effective treatments for cachexia. The capacity of BGM-2121 to alleviate this phenomenon implies a beneficial effect on patients suffering from cachexia. The results of serum chemistry demonstrated that BGM-2121 significantly reduces the secretion of hPTHrP (1-34) highlighting the regulatory role of BGM-2121 in PTHrP, a molecule known to regulate tumor progression and hypercalcemia. In summary, BGM-2121 exhibits promise as a potential therapeutic intervention for cachexia occurring independently of other medical conditions, or as a result of cancer, chemotherapy with or without immuno-oncology therapy, chronic obstructive pulmonary disease, chronic kidney disease, chronic heart failure, congestive heart failure, or sarcopenia, a severe condition characterized by muscle and fat depletion in advanced malignancies.

It will be understood that the above description of embodiments is given by way of example only and that various modifications may be made by those with ordinary skill in the art. The above specifications, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

### ABSTRACT OF THE DISCLOSURE

Disclosed herein is a method of treating cachexia in a subject. According to some embodiments of the present disclosure, the method includes administering to the subject an effective amount of a recombinant antibody or a fragment thereof, which exhibits a binding affinity and a neutralizing effect on parathyroid hormone-related protein. According to certain embodiments of the present disclosure, the cachexia is caused by a parathyroid hormone-related protein (PTHrP)-expressing cancer, chronic kidney disease, kidney failure, heart failure, or tuberculosis.

What is claimed is:

1. A method of treating cachexia in a subject comprising administering to the subject an effective amount of a recombinant antibody or a fragment thereof, wherein the recombinant antibody or the fragment thereof comprises a heavy chain variable (VH) domain and a light chain variable (VL) domain, wherein the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3), wherein
the CDR-H1, CDR-H2 and CDR-H3 respectively comprise the amino acid sequences of “GYXFTDY” (SEQ ID NO: 1), “DTSDSY” (SEQ ID NO: 2), and “GDY”, and the X residue in SEQ ID NO: 1 is isoleucine (I) or threonine (T); and
the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4).

2. The method of claim 1, wherein
the CDR-H1, CDR-H2 and CDR-H3 respectively comprise the amino acid sequences of “GYIFTDY” (SEQ ID NO: 5), “DTSDSY” (SEQ ID NO: 2), and “GDY”; and
the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4).

3. The method of claim 2, wherein the VH and VL domains respectively comprise the amino acid sequences of SEQ ID NOs: 7 and 8.

4. The method of claim 2, wherein
the VH domain comprises the amino acid sequence of SEQ ID NOs: 9, 10, 11 or 12; and
the VL domain comprises the amino acid sequence of SEQ ID NOs: 13, 14 or 15.

5. The method of claim 4, wherein the VH and VL domains respectively comprise the amino acid sequences of SEQ ID NOs: 11 and 13.

6. The method of claim 1, wherein
the CDR-H1, CDR-H2 and CDR-H3 respectively comprise the amino acid sequences of “GYTFTDY” (SEQ ID NO: 6), “DTSDSY” (SEQ ID NO: 2), and “GDY”; and
the CDR-L1, CDR-L2 and CDR-L3 respectively comprise the amino acid sequences of “QSLLESDGKTY” (SEQ ID NO: 3), “LVS”, and “CQGTHFPWT” (SEQ ID NO: 4).

7. The method of claim 6, wherein the VH and VL domains respectively comprise the amino acid sequences of SEQ ID NOs: 16 and 17.

8. The method of claim 1, wherein the cachexia is cancer cachexia.

9. The method of claim 8, wherein the cancer cachexia is caused by a parathyroid hormone-related protein (PTHrP)-expressing cancer.

10. The method of claim 9, wherein the cancer is gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

11. The method of claim 1, wherein the subject is a human.


SEQUENCE LISTING (19 sequences)

SEQ ID NO: 1 - protein, 7 aa, synthetic construct
  VARIANT 3: note = X is I or T
GYXFTDY

SEQ ID NO: 2 - protein, 6 aa, synthetic construct
DTSDSY

SEQ ID NO: 3 - protein, 11 aa, synthetic construct
QSLLESDGKTY

SEQ ID NO: 4 - protein, 9 aa, synthetic construct
CQGTHFPWT

SEQ ID NO: 5 - protein, 7 aa, synthetic construct
GYIFTDY

SEQ ID NO: 6 - protein, 7 aa, synthetic construct
GYTFTDY

SEQ ID NO: 7 - protein, 112 aa, synthetic construct
QVQLQQPGAELGMPGASVKMSCKASGYIFTDYWMHWVKQRPGQGLEWIGAIDTSDSYSSYNQKFQGKATLTVDESSSTAYMQLSSLTSEDSAVYYCTLGDYWGQGTTLTVSS

SEQ ID NO: 8 - protein, 112 aa, synthetic construct
DVVMTQTPLTLSVTIGQPASMSCKSSQSLLESDGKTYLNWLLQRPGQSPKRLIYLVSKLDSGVPDRFTGSGSGTDFTLKISRVEAEDLGVYYCCQGTHFPWTFGGGTKLEIK

SEQ ID NO: 9 - protein, 112 aa, synthetic construct
QVQLVQSGAEVKKPGASVKVSCKASGYIFTDYWMHWVRQAPGQGLEWMGAIDTSDSYSSYNQKFQGRVTMTRDTSTSTVYMELSSLRSEDTAVYYCTLGDYWGQGTTVTVSS

SEQ ID NO: 10 - protein, 112 aa, synthetic construct
QVQLVQSGAEVKKPGASVKVSCKASGYIFTDYWMHWVRQAPGQGLEWIGAIDTSDSYSSYNQKFQGRATMTVDTSTSTVYMELSSLRSEDTAVYYCTLGDYWGQGTTVTVSS

SEQ ID NO: 11 - protein, 112 aa, synthetic construct
QVQLVQSGAEVKKPGASVKVSCKASGYIFTDYWMHWVKQAPGQGLEWIGAIDTSDSYSSYNQKFQGRATMTVDTSTSTVYMELSSLRSEDTAVYYCTLGDYWGQGTTVTVSS

SEQ ID NO: 12 - protein, 112 aa, synthetic construct
QVQLVQSGAEVKKPGASVKVSCKASGYIFTDYWMHWVKQAPGQGLEWIGAIDTSDSYSSYNQKFQGRATMTVDESTSTVYMELSSLRSEDTAVYYCTLGDYWGQGTTVTVSS

SEQ ID NO: 13 - protein, 112 aa, synthetic construct
DVVMTQSPLSLPVTLGQPASISCKSSQSLLESDGKTYLNWFQQRPGQSPRRLIYLVSKLDSGVPDRFSGSGSGTDFTLKISRVEAEDVGVYYCCQGTHFPWTFGGGTKLEIK

SEQ ID NO: 14 - protein, 112 aa, synthetic construct
DVVMTQSPLSLPVTLGQPASISCKSSQSLLESDGKTYLNWLLQRPGQSPRRLIYLVSKLDSGVPDRFSGSGSGTDFTLKISRVEAEDVGVYYCCQGTHFPWTFGGGTKLEIK

SEQ ID NO: 15 - protein, 112 aa, synthetic construct
DVVMTQSPLSLPVTLGQPASMSCKSSQSLLESDGKTYLNWLLQRPGQSPKRLIYLVSKLDSGVPDRFSGSGSGTDFTLKISRVEAEDVGVYYCCQGTHFPWTFGGGTKLEIK

SEQ ID NO: 16 - protein, 112 aa, synthetic construct
QVQLQQPGAELVMPGASVKMSCKASGYTFTDYWMHWVKQRPGQGLEWIGALDTSDSYASYNQRFKGKATLTVDASSSTAYMQLSSLTSEDSAVYYCTLGDYWGQGTTLTVSS

SEQ ID NO: 17 - protein, 112 aa, synthetic construct
DVVMTQTPLTLSVTFGQPASISCKSSQSLLESDGKTYLNWLLQRPGQSPKRLIYLVSKLDSGVPDRFTGSGSGTDFTLKISRVEAEDLGVYYCCQGTHFPWTFGGGTKLEIK

SEQ ID NO: 18 - protein, 330 aa, synthetic construct [sequence omitted]

SEQ ID NO: 19 - protein, 107 aa, synthetic construct [sequence omitted]